(19) United States
(12) Patent Application Publication
(10) Pub. No.: US 2022/0238225 A1
(43) Pub. Date: Jul. 28, 2022

(54) Systems and Methods for AI-Enabled Instant Diagnostic Follow-Up

(71) Applicant: Google LLC, Mountain View, CA (US)

(72) Inventors: Marcin Tomasz Sieniek, Mountain View, CA (US); Sunny Jansen, Mountain View, CA (US); Krishnan Eswaran, Mountain View, CA (US); Shruthi Prabhakara, Sunnyvale, CA (US); Daniel Shing Shun Tse, San Francisco, CA (US); Scott Mayer McKinney, Oakland, CA (US)

(21) Appl. No.: 17/321,734

(22) Filed: May 17, 2021

Related U.S. Application Data
(60) Provisional application No. 63/140,494, filed on Jan. 22, 2021.

(51) Int. Cl. G16H 50/20; G16H 50/30; G16H 40/20; G06T 7/00

(52) U.S. Cl. CPC G16H 50/20; G16H 50/30; G16H 40/20; G06T 2207/20084; G06T 2207/30068; G06T 2207/20081; G06T 7/0012

(57) ABSTRACT

Systems and methods for artificial intelligence enabled instant diagnostic follow-up can provide efficiency to the medical diagnosis process by providing multiple examinations in the same medical visit for medical workflows that previously required multiple visits. The systems and methods can provide further medical benefit by implementing triaging systems to ensure the most urgent cases are seen immediately.

### RELATED APPLICATIONS

This application claims priority to and the benefit of U.S. Provisional Patent Application No. 63/140,494, filed Jan. 22, 2021. U.S. Provisional Patent Application No. 63/140,494 is hereby incorporated by reference in its entirety.

### FIELD

The present disclosure relates generally to artificial intelligence enabled triaging and workflow management. More particularly, the present disclosure relates to providing an urgency score for patients needing a follow-up visit based on the processing of medical data with a machine-learned model, which generates predictions that can be used for generating the urgency score.

### BACKGROUND

In medical workflows, frequently multiple steps lead to a diagnosis. For non-emergent diseases, a diagnosis can span multiple visits in a healthcare facility, leading to delays in diagnosis, and poor customer experience. This can include follow-up examination after follow-up examination.

Delays can be caused by: the extensive time it takes to interpret the results of an image or specimen taken at a prior visit; unavailability or lack of expertise of staff to interpret the results of an image or specimen; and/or scheduling difficulties with respect to the subsequent visit. Patients are often called in to have a quick follow-up examination that could have easily been completed during the previous visit if the original examination data was processed immediately. However, efficiency issues related to human processing often create a time delay hurdle.

Moreover, serious cases may be overshadowed by a large volume of normal ones, which can cause diseases to go undiagnosed for weeks or months. Purely chronological follow-up ordering can see those with low urgency illnesses being prioritized the same as those with life threatening diseases.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method. The method can include obtaining, by a computing device, initial medical data. In some implementations, the method can include processing, by the computing device, the initial medical data with a machine-learned model to generate a probability score based at least in part on a determined probability of a positive test result. The method can include determining, by the computing device, if the probability score is above a positive threshold and providing, by the computing device, a suggested next action based at least in part on whether the probability score is above the positive threshold.

Another example aspect of the present disclosure is directed to a computing system for ranking urgency and next patient up. The system can include one or more processors and one or more non-transitory computer readable media that collectively store instructions that, when executed by the one or more processors, cause the computing system to perform operations. The operations can include obtaining one or more predictions for a patient. The operations can include processing the one or more predictions to generate an urgency score. The urgency score can be descriptive of an urgency for a follow-up. In some implementations, the operations can include determining a ranking for the patient based at least in part on the urgency score. The ranking can determine when a patient is seen for a follow-up in relation to other patients.

Another example aspect of the present disclosure is directed to one or more non-transitory computer readable media that collectively store instructions that, when executed by one or more processors, cause a computing system to perform operations. The operations can include obtaining examination data. In some implementations, the examination data can include medical data from an examination of a patient. The operations can include generating one or more predictions from one or more machine-learned models based on the examination data. The operations can include modifying a medical workflow for the patient based on the one or more predictions.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system that performs triaging according to example embodiments of the present disclosure.

FIG. 1B depicts a block diagram of an example computing device that performs triaging according to example embodiments of the present disclosure.

FIG. 1C depicts a block diagram of an example computing device that performs triaging according to example embodiments of the present disclosure.

FIG. 2 depicts a block diagram of an example triage model according to example embodiments of the present disclosure.

FIG. 3 depicts an illustration of an example suggested action according to example embodiments of the present disclosure.

FIG. 4 depicts an illustration of an example ranking model according to example embodiments of the present disclosure.

FIG. 5 depicts a block diagram of an example triage model according to example embodiments of the present disclosure.

FIG. 6 depicts a flow chart diagram of an example method to perform triaging according to example embodiments of the present disclosure.

FIG. 7 depicts a flow chart diagram of an example method to perform ranking according to example embodiments of the present disclosure.

FIG. 8 depicts a flow chart diagram of an example method to perform triaging according to example embodiments of the present disclosure.

Reference numerals that are repeated across plural figures are intended to identify the same features in various implementations.

### DETAILED DESCRIPTION

### Overview

Generally, the present disclosure is directed to systems and methods for artificial intelligence enabled triaging or other medical process or workflow management. The systems and methods disclosed herein can provide for quicker diagnostic follow-up by determining the likelihood of positive results with machine-learned models. Systems and methods for diagnostic follow-up can shorten the time until final diagnosis and can add efficiency to many medical diagnosis processes. The systems and methods can lead to less office visits, which can lessen the time between a first check-up and a final diagnosis. For example, the systems and methods can help provide a suggested next action to have a follow-up exam before the patient leaves, which can cut-out a second visit before the patient even leaves. In some implementations, the systems and methods can include obtaining an initial set of medical data, which can include answers to a questionnaire, an image, a specimen, or testing data. The systems and methods can further include processing the initial medical data with a machine-learned model to generate a probability score. The probability score can be based at least in part on a determined probability of a positive test result. The systems and methods can then determine if the probability score meets a positive threshold, in which the positive threshold can be based on a level of certainty for a positive test result which would require further testing. If the positive threshold is met, the systems and methods may suggest a follow-up test. The suggestion of a follow-up can be a direct recommendation of a follow-up test or can be a prioritization of immediate human review, which can lead to a human expert immediately reviewing the initial medical data to determine whether to recommend a follow-up test. If the positive threshold is not met, the systems and methods may suggest allowing the patient to leave or informing the patient of the results.

The systems and methods can be used for a variety of diagnostic fields, including but not limited to mammography. The system can begin with a patient visiting a medical facility for a medical exam (e.g., a medical imaging exam or a biopsy). An image or specimen can be collected, and the patient can be asked to wait at the medical facility. While the patient is still at the medical facility, a machine-learned model can process the image or specimen. The artificial intelligence model may optimize for any of the follow-up stages in the workflow, including mortality rate, disease state, likelihood of human experts to recall a patient for further diagnostic stages, etc. In some implementations, in cases where the exam has a very high probability of being negative, the patient may be informed about the result. In cases where the exam has a higher probability of having a positive result a notification (e.g., mobile call, text, email, page, etc.) can be sent in order to alter the patient's journey during the visit.

In some implementations, the systems and methods can begin with the intake of initial medical data. The initial medical data can include but is not limited to questionnaire data, image data, or examination data. Examination data can include x-rays, mammograms, EKG results, or any form of examination data. The examination data can include a specimen taken from the patient. The specimen can be a biological specimen taken for specific diagnosis or general diagnosis.

The initial medical data can then be processed by a machine-learned model to generate predictions. In some implementations, the machine-learned model may be trained to generate predictions related to a specific medical field or a specific disease, illness, or diagnosis. For example, in some implementations, the machine-learned model may be trained for determining a likelihood of breast cancer based on the initial medical data. In some implementations, the machine-learned model can be trained to generate general predictions related to a variety of possibilities related to a variety of diagnosable fields. The machine-learned models may be trained to generate predictions related to the general field of medical pathology.

In some implementations, the one or more predictions can include predictions related to the urgency of a follow-up and a prediction of the likelihood of a positive test result. The predictions can include whether a follow-up is advisable, whether a human expert would advise a follow-up, an initial diagnosis, a binary prediction on an illness, a time to event, a disease state, or another prediction related to the processed medical data.

The one or more predictions can be used to modify the medical workflow. The modification of the workflow can include providing suggestions for next actions based on the one or more predictions. Moreover, the modification of the medical workflow can include generating an urgency score for the patient based on the one or more predictions and modifying a triaging ranking for the patient relative to one or more other patients based on the urgency score for the patient. In some implementations, modifying the medical workflow can include automatically scheduling a same day follow-up test based on the one or more predictions. Furthermore, in some implementations, modifying the medical workflow can include classifying the patient into an urgent group or a non-urgent group.

In some implementations, the systems and methods can include a ranking scheme for determining a ranking for testing to determine which patients need to stay for further testing and which patients can be sent home.

In some implementations, an image or specimen can be prioritized for an interpretation. If the interpretation indicates a high probability of being positive, the patient can get routed to the next stage of the diagnostic funnel. If the interpretation indicates a low probability of being positive, the patient can be instructed to go home. Patient's image or specimen can continue through the usual interpretation queue. In some implementations, the interpretation queue may be ordered by a suspicion score, an urgency score, or a probability score. The ordering by score can take place above a certain score threshold, after which the cases can be ordered chronologically, to avoid starving out the least suspicious cases.

In some implementations, systems and methods for ranking urgency and next patient up can include obtaining one or more predictions for a patient. The predictions can include a likelihood of a positive test result for a diagnosis. The predictions can also include a mortality rate for the diagnosis, a possible disease state, a gestation period for a disease, the patient's susceptibility, or other factors that can relate to urgency of a potential follow-up. The one or more predictions can be processed to generate a score. The score can be descriptive of an urgency for a follow-up test or visit. The score can then be used to determine a ranking for the patient. The ranking can then be used to decide when a follow-up visit for the patient will occur in relation to other patients.

Ranking may include determining whether the score is above or below a threshold. If the score is above the threshold, the patient can be ranked by the urgency of the follow-up. In this case, the patient can be ranked above patients with less urgency and below patients with higher urgency. If the score is below a threshold, the patient can be ranked chronologically, where the patient is ranked above patients with a later visit time and below patients with an earlier visit time. In some implementations, the ranking system can be flipped, wherein the higher urgency scores are ranked chronologically, and the lower urgency scores are ranked by score. In some implementations, a higher urgency score can be indicative of a higher urgency level. In some implementations, a lower urgency score can be indicative of a higher urgency level.

The threshold can be based on a variety of factors and can be manually set or automatically set based on the variety of factors. The threshold can be based at least in part on time of individual follow-ups, hospital occupancy, hospital capacity, a set urgency level, or other factors. The threshold can be set to ensure that all patients are seen in a timely fashion, while ensuring the more urgent patients are seen as soon as possible.

The systems and methods can be run iteratively for each step of the diagnostic process. In some implementations, the iterative nature can occur multiple times in one visit, such that a patient may receive a follow-up examination or multiple follow-up examinations during the initial visit.

In some implementations, the systems and methods can be used to provide immediate feedback from a medical visit. For example, the systems and methods can be used to provide an immediate binary feedback of whether the patient should go home or receive an immediate follow-up examination. The feedback may be delivered via a human to human exchange or via any communication medium (e.g., text message, email, phone call, page, etc.). In some implementations, the system may automatically inform the patient without further selection from human interaction.

The systems and methods disclosed herein can include training the machine-learned models with training sets that include images or specimens paired with long term outcomes. The models may be iteratively trained, and the training can include supervised learning. The supervised learning may continue until a certain level of accuracy is achieved. In some implementations, the machine-learned model can be trained with data on how many negatives can occur before a positive result, mortality rate, disease state, and likelihood of recall for each particular disease or illness.

The probability score and the urgency score can be calculated in a variety of processes that can include, but is not limited to, weighting of scores, summation of scores, an individual score, or a combination of weighting and summation. In some implementations, the urgency score can be calculated by a set of partitioned pools that each intake and produce a score. The scores can then be summed to provide a final urgency score. Alternatively, the scores may be aggregated or multiplied together.

The data used by the systems and models (e.g., for training and/or inference) described herein can be de-identified data. For example, personally identifiable information, such as location, name, exact birth date, contact information, biometric information, facial photographs, etc. can be eliminated from the records prior to being transmitted to and/or utilized by the models and/or a computing system including the models. For example, the data can be de-identified to protect identity of individuals and to conform to regulations regarding medical data, such as HIPAA, such that no personally identifiable information (e.g., protected health information) is present in the data used by the models and/or used to train the models.

Further to the descriptions above, a user may be provided with controls allowing the user to make an election as to both if and when systems, programs, or features described herein may enable collection of user information (e.g., examination data). In addition, certain data may be treated in one or more ways before it is stored or used, so that personally identifiable information is removed. For example, a user's identity may be treated so that no personally identifiable information can be determined for the user. Thus, the user may have control over what information is collected about the user, how that information is used, and what information is provided to the user.

The systems and methods of the present disclosure provide a number of technical effects and benefits. As one example, the system and methods can generate one or more predictions that can lead to fewer follow-up visits and quicker diagnosis. The systems and methods can further be used to provide a triage for ranking patients to allow for the most urgent follow-ups to occur first. Furthermore, the systems and methods can enable the medical providers to provide a follow-up examination for a patient before the patient even leaves the preliminary examination.

Another technical benefit of the systems and methods of the present disclosure is the ability to automatically notify a patient of a need for a follow-up or the notification giving a patient the okay to go home.

With reference now to the Figures, example embodiments of the present disclosure will be discussed in further detail.

### Example Devices and Systems

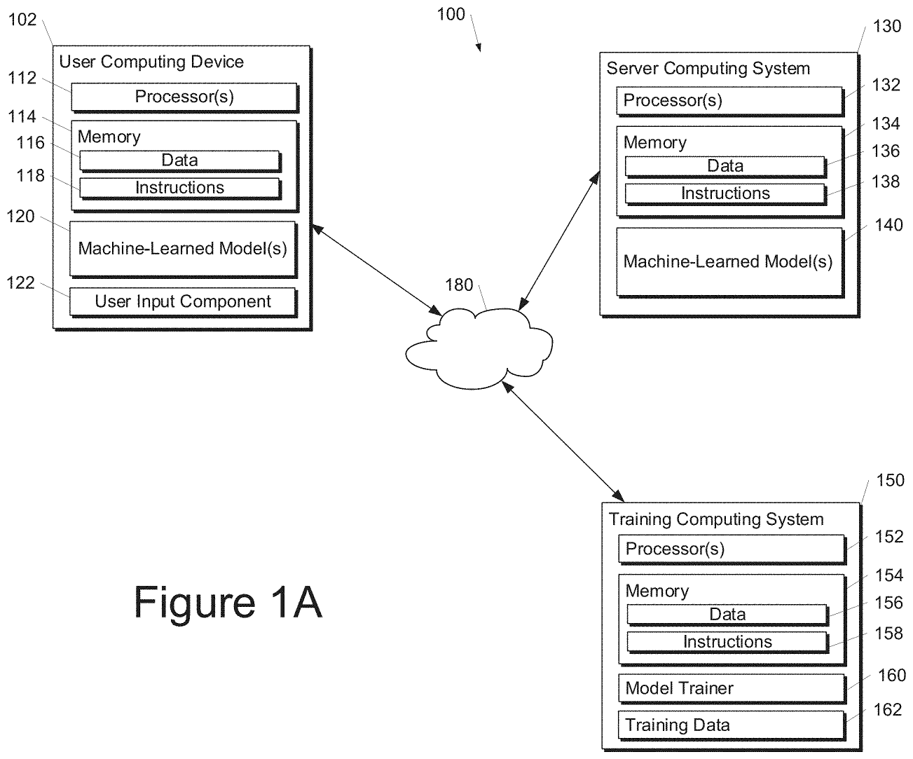

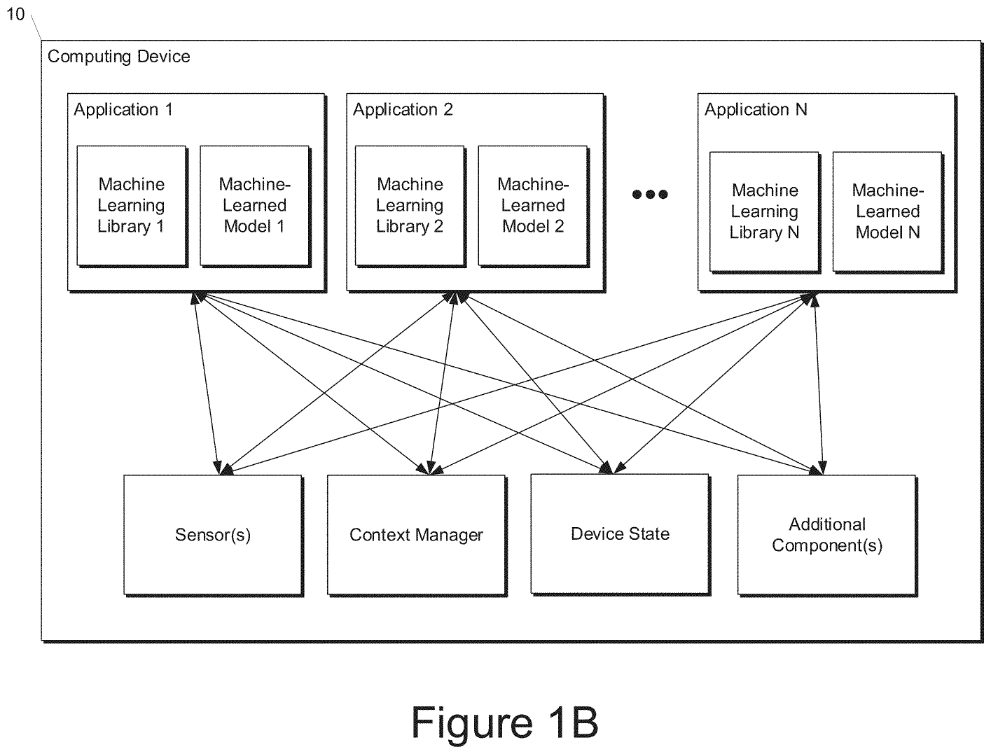

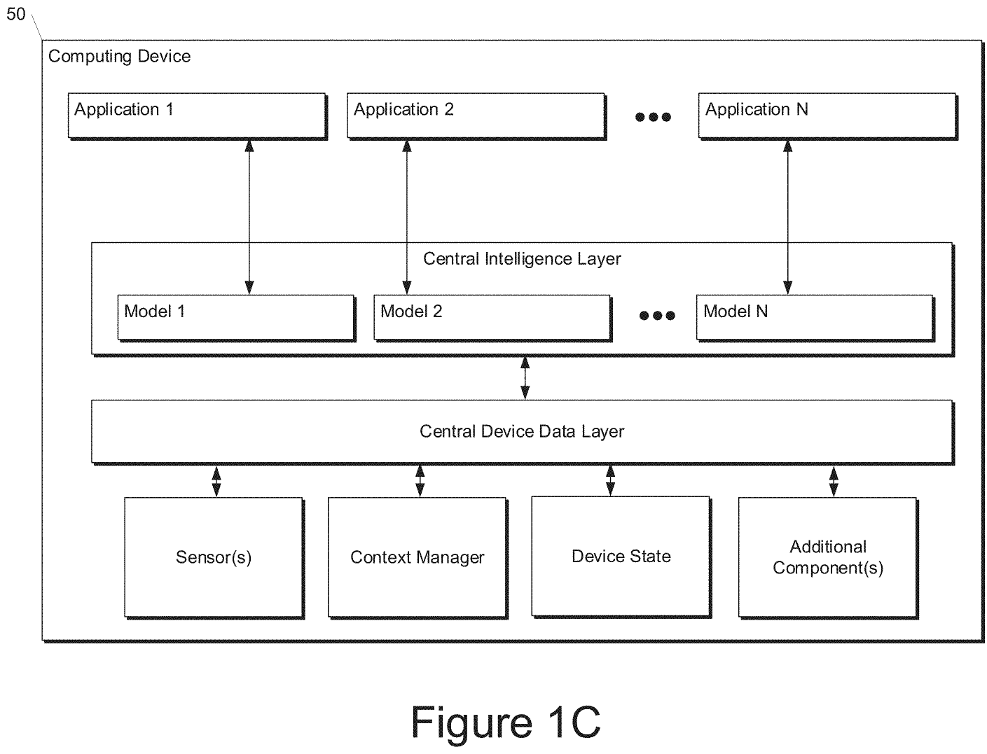

FIG. 1A depicts a block diagram of an example computing system 100 that performs triaging according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

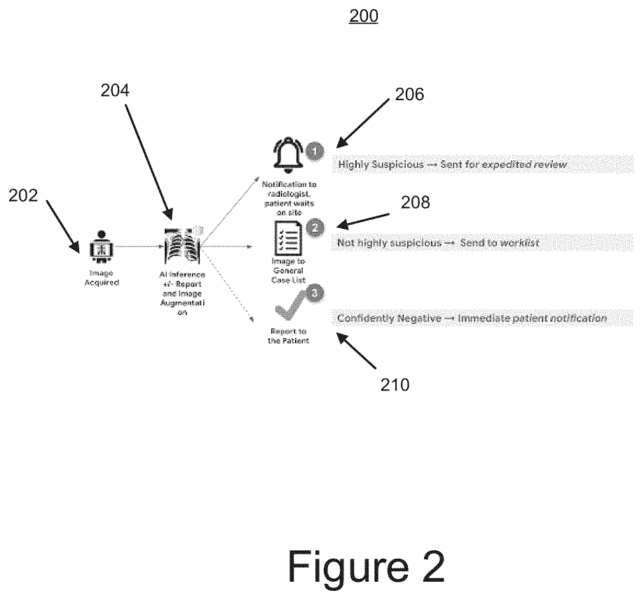

In some implementations, the user computing device 102 can store or include one or more triage models 120. For example, the triage models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example triage models 120 are discussed with reference to FIGS. 2, 4-6, & 8.

In some implementations, the one or more triage models 120 can be received from the server computing system 130 over network 180, stored in the user computing device memory 114, and then used or otherwise implemented by the one or more processors 112. In some implementations, the user computing device 102 can implement multiple parallel instances of a single triage model 120 (e.g., to perform parallel triaging across multiple instances of patients getting examined).

More particularly, the one or more triage models can be used to process examination data to generate one or more predictions. The one or more predictions can be used to generate an urgency score. In some implementations, the one or more predictions can include the urgency score. In some implementations, the one or more predictions can include a probability score indicative of a probability of a positive test result. The urgency score and/or the urgency score can be used to determine a suggested next action for the medical workflow. For example, in some cases, if the probability score is above a positive threshold, the patient may be contacted to have an immediate follow-up examination. If a positive threshold is not met, the patient may be notified that they can leave the medical facility.

In some implementations, the urgency score can be used to determine when the patient will be seen for a follow-up. For example, a first patient with a high likelihood of having a severe disease can be prioritized over a second patient with a low likelihood of having a minor illness, and therefore, the first patient may be assigned a follow-up visit before the second patient regardless of visit time. However, in some implementations, the triage model can be accompanied by or can include a ranking model, which in some instances may have a multiple pronged method for ranking. In some implementations, the ranking model can include ranking based on the urgency score for scores above a certain threshold and can include ranking based on the time of the visit for scores below a certain threshold. The threshold can be based on a threshold urgency score or can alternatively be based on a number of patients.

Additionally or alternatively, one or more triage models 140 can be included in or otherwise stored and implemented by the server computing system 130 that communicates with the user computing device 102 according to a client-server relationship. For example, the triage models 140 can be implemented by the server computing system 140 as a portion of a web service (e.g., a medical waitlist service). Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned triage models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIGS. 2, 4-6, & 8.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the triage models 120 and/or 140 based on a set of training data 162. The training data 162 can include, for example, medical data (e.g., images, text, etc.) paired with a diagnosis. In some implementations, the training data 162 can further include information related to the mortality rate, the disease state, the examinations needed, the likelihood of first test positive result, and/or the gestation period for the diagnosis. In some implementations, the machine-learned model can be trained on the pairings and can then be trained with supervised learning. A human expert can ensure the correct outcomes are being reinforced and ensure incorrect biases do not form from the initial training set. In some implementations, the supervised training can continue indefinitely, and in some implementations, the supervised training can be completed when a certain level of accuracy is obtained.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the input includes visual data, and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 1B depicts a block diagram of an example computing device 10 that performs according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

### Example Model Arrangements

FIG. 2 depicts a block diagram of an example triage model 200 according to example embodiments of the present disclosure. In some implementations, the triage model 200 is trained to receive a set of input data 202 descriptive of initial medical data or examination data and, as a result of receipt of the input data 202, provide output data 206, 208, or 210 that includes one or more predictions and/or a suggested next action. Thus, in some implementations, the triage model 200 can include an inference model 204 that is operable to infer a probability of a positive or negative result based at least in part on the input data 202.

More particularly, in FIG. 2, the input data 202 is medical image data, which can include a camera photo, an x-ray, or any other form of imagery. In this implementation, the input data 202 is processed by the inference model 204 to generate output data. The inference model 204 can utilize artificial intelligence to determine a probability of a positive or negative result. Moreover, in some implementations, the inference model 204 may augment an input image for classification, identification, or for aiding a human expert. The output of the inference model 204 can include various suggested actions including but not limited to further examination 206, adding to worklist 208, or notifying the patient 210. In some implementations, the inference model may process the input data 202, determine a score, and then use the score to determine which action will be suggested. The score may be a probability score related to the likelihood of a positive test result, or alternatively, the score may be an urgency score related to various factors that can include a mortality rate of a potential diagnosis, a disease state of the potential diagnosis, and/or health of the patient. The suggested action can be determined based on what grouping the score falls into. For example, the triage model 200 may be trained to suggest one of three actions. If the score falls into the confidently negative grouping, the suggested action can be notifying the patient 210 of a negative result. If the score falls into the not highly suspicious grouping, the suggested action can be adding the patient's case to the worklist 208. Lastly, if the score falls into the highly suspicious grouping, the suggested action can be immediate further examination 206.

In some implementations, the groupings can be based at least in part on determined thresholds. For example, the groupings can be based on the level of likelihood of a positive result.

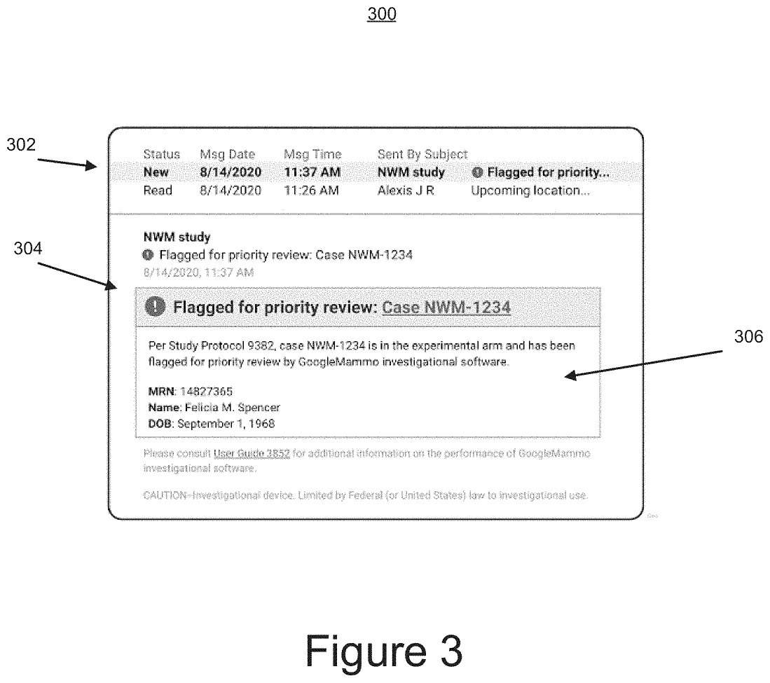

FIG. 3 depicts an illustration of an example suggested action 300 according to example embodiments of the present disclosure. In some implementations, the example suggestion 300 can include the proposed action 304 descriptive of a modification to the medical workflow, and, with the proposed action 304, the suggested action can include reasoning 306 that is descriptive of a reason for the suggested action. Thus, in some implementations, the suggested action 300 can include a catalog 302 that is operable to show a list of previous suggested actions or results for the patient or a plurality of patients.

The illustration depicted in FIG. 3 includes an example suggested action 300 in which priority review is suggested based on the processing completed by the triage model. In this implementation, two matters are listed in the catalog 302, including the open matter with the proposed action 304 for priority review. The open matter can include the proposed action 304 and the reasoning 306 for the proposed action 304. For example, in FIG. 3, the reasoning 306 for priority review is further accompanied by patient information. The reasoning 306 can include a reference to a case study, a reference to a set of training data, an augmented image, or another form of data interpretable by a human expert.

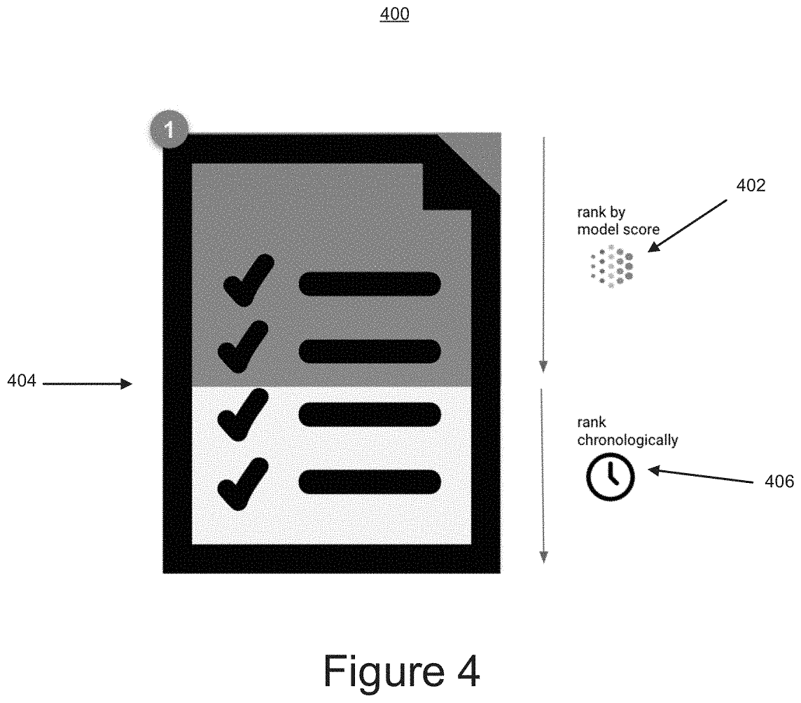

FIG. 4 depicts an illustration of an example ranking model 400 according to example embodiments of the present disclosure. In some implementations, the ranking model 400 is trained to rank a set of input data based on an urgency score 402 and/or chronologically 406. Thus, in some implementations, the ranking model 400 can include a threshold 404 that is operable to determine whether the input data is ranked based on the urgency score 402 or chronologically 406.

The ranking model 400 of FIG. 4 includes a ranking based on urgency scores and a ranking based on the chronology of the patients' visits. In this implementation, a triage model has processed a set of initial medical data to generate one or more predictions. The one or more predictions can be processed by the ranking model 400 to generate urgency scores for each set of predictions. The urgency scores can then be used to rank the urgency of each patient's case. The ranking model 400 can then determine which cases are above and below a threshold 404. If the case is above the threshold 404, the case may be ranked based on the urgency score 402, in which the case is ranked below cases with higher urgency scores and above cases with lower urgency scores. If the case is below the threshold 404, the case may be ranked based on the chronology 406 of the patient's visit. The threshold 404 can be a determined threshold urgency score based on a determined urgency level, a certain number of patients, or a variety of other factors.

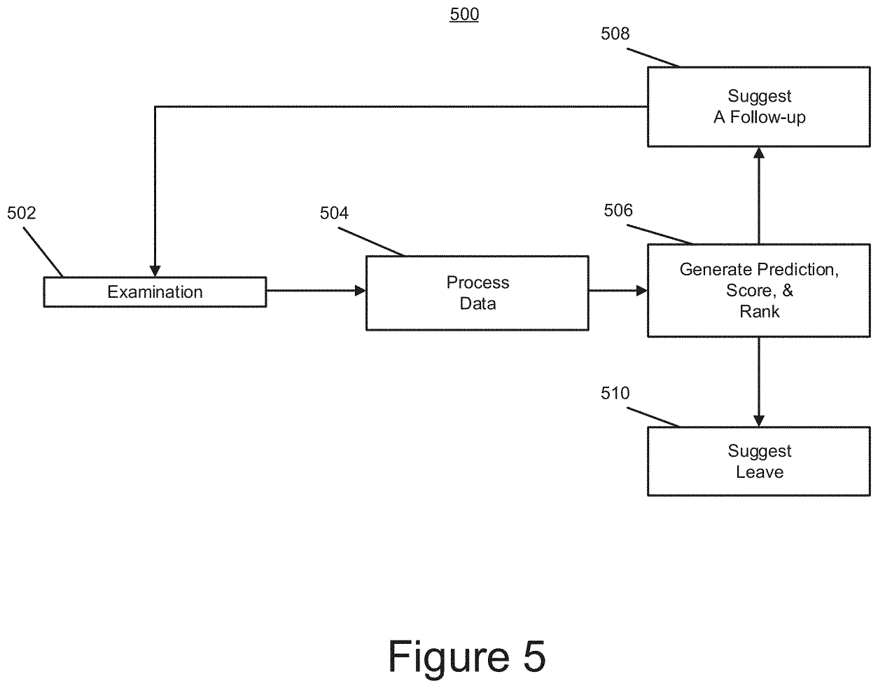

FIG. 5 depicts a block diagram of an example triage model 500 according to example embodiments of the present disclosure. The triage model 500 is similar to the triage model 200 of FIG. 2 except that the triage model 500 further includes an iterative function to convey the possible iterative nature of some implementations.

The triage model 500 can include intaking examination data 502, processing the examination data 504, and generating predictions, a score, and/or a rank 506. The generated predictions, score, and/or rank can then be used to determine a suggested action. The suggested action can include suggesting a follow-up 508 or suggesting the patient leave 510. The process can be repeated after each examination until a diagnosis is determined. For example, a patient may go in for a breast cancer screening. The patient may be examined with a mammography machine to generate examination data 502 in the form of a medical image (e.g., a mammogram). The medical image can be processed 504 to generate one or more predictions, a probability score, and an urgency score. The one or more predictions can include a possible disease state, a predicted size of a mass, or a predicted location for the cancerous growth. In some implementations, the probability score can be descriptive of a determined likelihood of breast cancer based on the processing of the medical image. The urgency score can be determined by a variety of factors including the likelihood of breast cancer, a predicted cancerous stage, a predicted size, and the age and health of the patient. One or both of the scores can then be used to determine a suggested action.

In some implementations, if the probability score is above a threshold probability, the suggested action can be a follow-up examination 508. The follow-up examination can be completed, and the process may start over with the new examination data being processed. In some implementations, the follow-up examination can occur during the same medical visit as the original examination. Alternatively, if the probability score is below a threshold, the patient may be told they can leave the facility 510. The examination data can be continued to be examined, or, depending on the score, the patient may be notified of a negative result.

In some implementations, if the urgency score is above a threshold urgency, the suggested action can be a follow-up examination 508. The follow-up examination can be completed, and the process may start over with the new examination data being processed. In some implementations, the follow-up examination can occur during the same medical visit as the original examination. Alternatively, if the urgency score is below a threshold, the patient may be told they can leave the facility 510. The patient may be added to a waitlist or scheduled for a future visit based on the lower urgency score.

### Example Methods

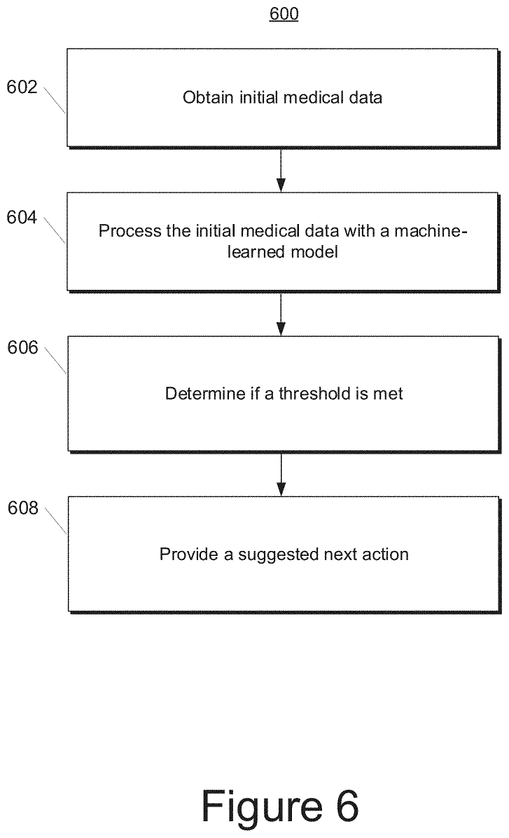

FIG. 6 depicts a flow chart diagram of an example method to perform according to example embodiments of the present disclosure. Although FIG. 6 depicts steps performed in a particular order for purposes of illustration and discussion, the methods of the present disclosure are not limited to the particularly illustrated order or arrangement. The various steps of the method 600 can be omitted, rearranged, combined, and/or adapted in various ways without deviating from the scope of the present disclosure.

At 602, a computing system can obtain initial medical data. The initial medical data can include answers to a questionnaire, one or more images, one or more specimens, and/or textual data. For example, the initial medical data can be a mammogram.

At 604, the computing system can process the initial medical data with a machine-learned model. In some implementations, the machine-learned model can output a probability score based at least in part on a determined probability of a positive test result. For example, the probability score can be indicative of how likely the processed mammogram will result in a cancer diagnosis.

At 606, the computing system can determine if a threshold is met. In some implementations, a threshold can be met by having a score above a threshold, or alternatively, a threshold can be met by having a score below a threshold. The threshold may be a positive threshold, in which any probability score above the positive threshold has a high likelihood of a positive result.

At 608, the computing system can provide a suggested next action. The suggested action can be based at least in part on whether the probability score is above the positive threshold. In some implementations, if the probability score is above the positive threshold, the suggested action may be a follow-up examination. Alternatively, if the probability score is below the positive threshold, the suggested action may be letting the patient go home or notifying the patient of a negative result.

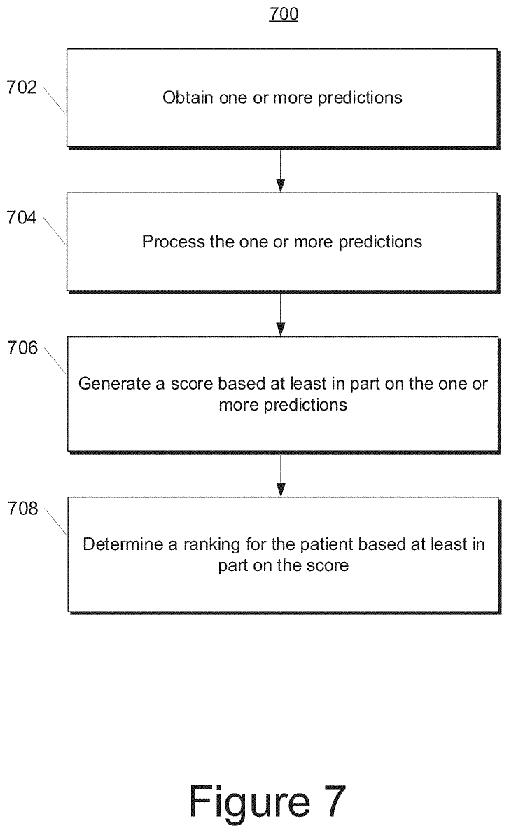

FIG. 7 depicts a flow chart diagram of an example method to perform according to example embodiments of the present disclosure. Although FIG. 7 depicts steps performed in a particular order for purposes of illustration and discussion, the methods of the present disclosure are not limited to the particularly illustrated order or arrangement. The various steps of the method 700 can be omitted, rearranged, combined, and/or adapted in various ways without deviating from the scope of the present disclosure.

At 702, a computing system can obtain one or more predictions. The one or more predictions can be predictions related to the medical state of a patient. In some implementations, the one or more predictions can include a probability score, a possible disease state, a mortality rate, or other predictions related to a possible diagnosis.

At 704, the computing system can process the one or more predictions. The predictions may be processed by one or more machine-learned models.

At 706, the computing system can generate a score based at least in part on the one or more predictions. The score may be an urgency score generated by processing the one or more predictions. In some implementations, the urgency score can be descriptive of an urgency for a follow-up visit. The urgency may be based at least in part on a severity of a potential disease or illness, the likelihood of a positive result, a disease state, a gestation period, a mortality rate, or the health of the patient.

At 708, the computing system can determine a ranking for the patient based at least in part on the score. In some implementations, the ranking for the patient can be made in relation to other patients. The ranking may be purely on the urgency score as compared to the urgency score of other patients. In some implementations, the ranking can be a hybrid ranking based on a threshold, in which all scores above the threshold are ranked based on the urgency score, and all scores below the threshold are ranked based on the time of the patient's visit.

The ranking can be used to determine an order of a follow-up visit for the patient in relation to other patients.

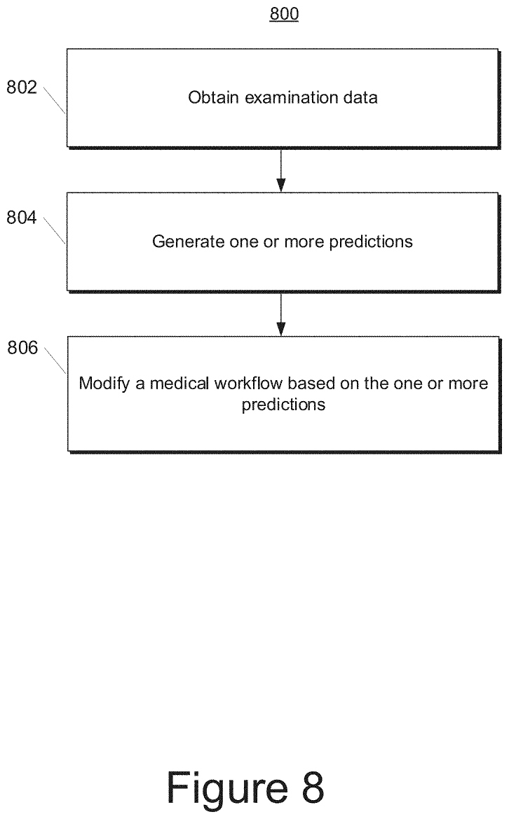

FIG. 8 depicts a flow chart diagram of an example method to perform according to example embodiments of the present disclosure. Although FIG. 8 depicts steps performed in a particular order for purposes of illustration and discussion, the methods of the present disclosure are not limited to the particularly illustrated order or arrangement. The various steps of the method 800 can be omitted, rearranged, combined, and/or adapted in various ways without deviating from the scope of the present disclosure.

At 802, a computing system can obtain examination data. Examination data can include image data, text data, and/or organic data. In some implementations, the examination data can include medical data descriptive of an examination of a patient.

At 804, the computing system can generate one or more predictions. The one or more predictions can be generated by processing the examination data with one or more machine-learned models. In some implementations, the one or more machine-learned models may be trained for general pathology, and in some implementations, the one or more machine-learned models may be trained for a specific medical field (e.g., oncology).

At 806, the computing system can modify a medical workflow based on the one or more predictions. Modifying the medical workflow can include providing a suggested next action, which can include a follow-up examination, adding the patient to a worklist, or notifying the patient of the result. In some implementations, the medical workflow can be modified while the patient is still at the medical facility, which can allow the patient to receive a follow-up examination the same day as the initial examination.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A computer-implemented method, the method comprising:
obtaining, by a computing device, initial medical data;
processing, by the computing device, the initial medical data with a machine-learned model to generate a probability score based at least in part on a determined probability of a positive test result;
determining, by the computing device, if the probability score is above a positive threshold; and
providing, by the computing device, a suggested next action based at least in part on whether the probability score is above the positive threshold.

2. The method of claim 1, wherein the initial medical data comprises image data.

3. The method of claim 1, wherein the initial medical data comprises a mammogram.

4. The method of claim 1, wherein the machine-learned model is trained for determining a likelihood of breast cancer based on the initial medical data.

5. The method of claim 1, wherein the initial medical data comprises a biological specimen collected from the patient.

6. A computing system for ranking urgency and next patient up, the system comprising:
one or more processors;
one or more non-transitory computer readable media that collectively store instructions that, when executed by the one or more processors, cause the computing system to perform operations, the operations comprising:
obtaining one or more predictions for a patient;
processing the one or more predictions to generate an urgency score, wherein the urgency score is descriptive of an urgency for a follow-up;
determining a ranking for the patient based at least in part on the urgency score; and
wherein the ranking determines when a patient is seen for a follow-up in relation to other patients.

7. The computing system of claim 6, further comprising: determining whether the urgency score is above a threshold; and
in response to determining the urgency score is above the threshold, ranking the patient above patients with lower urgency and below patients with higher urgency.

8. The computing system of claim 6, further comprising: determining whether the urgency score is above a threshold; and
in response to determining the urgency score is below the threshold, ranking the patient above patients with a later visit time and below patients with an earlier visit time.

9. The computing system of claim 6, wherein the urgency score is based at least in part on mortality rate.

10. The computing system of claim 6, wherein the urgency score is based at least in part on disease state.

11. One or more non-transitory computer readable media that collectively store instructions that, when executed by one or more processors, cause a computing system to perform operations, the operations comprising:
obtaining examination data, wherein the examination data comprises medical data from an examination of a patient;
generating one or more predictions from one or more machine-learned models based on the examination data; and
modifying a medical workflow for the patient based on the one or more predictions.

12. The one or more non-transitory computer readable media of claim 11, wherein modifying comprises:
generating an urgency score for the patient based on the one or more predictions; and
modifying a triaging ranking for the patient relative to one or more other patients based on the urgency score for the patient.

13. The one or more non-transitory computer readable media of claim 11, wherein modifying comprises: automatically scheduling a same day follow-up test based on the one or more predictions.

14. The one or more non-transitory computer readable media of claim 11, wherein modifying comprises: classifying the patient into an urgent group or a non-urgent group.

15. The one or more non-transitory computer readable media of claim 11, wherein the one or more predictions comprise whether a follow-up visit is advisable.

16. The one or more non-transitory computer readable media of claim 11, wherein the one or more predictions comprise an initial diagnosis.

17. The one or more non-transitory computer readable media of claim 11, wherein the one or more predictions comprises a binary prediction on an illness.

18. The one or more non-transitory computer readable media of claim 11, wherein the one or more predictions comprise a time to event.

19. The one or more non-transitory computer readable media of claim 11, wherein the one or more predictions comprise whether a human expert would advise a follow-up.

20. The one or more non-transitory computer readable media of claim 11, the operations further comprising providing a notification to the patient.
